(19) United States
(12) Patent Application Publication
(10) Pub. No.: US 2022/0288155 A1
(43) Pub. Date: Sep. 15, 2022

(54) CXCR4 INHIBITOR FOR THE TREATMENT OF ACUTE RESPIRATORY DISTRESS SYNDROME AND VIRAL INFECTIONS

(71) Applicant: BioLineRx Ltd., Modiln (IL)

(72) Inventor: Gideon STEIN, Rosh HaAyin (IL)

(73) Assignee: BioLineRx Ltd., ModiIn (IL)

(21) Appl. No.: 17/827,832

(22) Filed: May 30, 2022

Related U.S. Application Data
(63) Continuation of application No. PCT/IL2021/050273, filed on Mar. 11, 2021.
(60) Provisional application No. 62/987,995, filed on Mar. 11, 2020.
(60) Provisional application No. 63/026,059, filed on May 17, 2020.
(60) Provisional application No. 63/106,419, filed on Oct. 28, 2020.

(51) Int. Cl. A61K 38/10; A61K 31/395; A61P 31/14; A61P 11/16

(52) U.S. Cl. CPC A61K 38/10; A61K 31/395; A61P 31/14; A61P 11/16

(57) ABSTRACT

A method of treating acute respiratory distress syndrome. The method comprising administering to a subject in need thereof a therapeutically effective amount of a CXCR4 inhibitor.

### RELATED APPLICATIONS

This application is a Continuation of PCT Patent Application No. PCT/IL2021/050273 having International filing date of Mar. 11, 2021, which claims the benefit of priority under 35 USC § 119(e) of U.S. Provisional Patent Application No. 63/106,419 filed on Oct. 28, 2020, U.S. Provisional Patent Application No. 63/026,059 filed on May 17, 2020 and U.S. Provisional Patent Application No. 62/987,995 filed on Mar. 11, 2020. The contents of the above applications are all incorporated by reference as if fully set forth herein in their entirety.

### SEQUENCE LISTING STATEMENT

The ASCII file, entitled 92750SequenceListing.txt, created on May 30, 2022, comprising 39,857 bytes, submitted concurrently with the filing of this application is incorporated herein by reference.

### FIELD AND BACKGROUND OF THE INVENTION

The present invention, in some embodiments thereof, relates to treatment of acute respiratory distress syndrome and viral infections.

Novel Coronavirus (2019-nCoV or COVID-19) is an emerging pathogen that was first identified in Wuhan, China in late December 2019. This virus is responsible for the ongoing outbreak that causes severe respiratory illness and pneumonia-like infection in humans. Due to the increasing number of cases in China and outside China, the WHO declared Coronavirus as a global health emergency. Inter-human transmission was reported in a few countries, including the United States. Neither an effective anti-viral nor a vaccine is currently available to treat this infection.

Middle East respiratory syndrome Coronavirus (MERS-CoV) has emerged on 2012 as another very infective Coronavirus, and to date no antiviral or therapeutic has been approved for treating patients. Since September 2012, 206 cases, including 86 deaths, have been attributed to infection with MERS-CoV. Currently, supportive care remains the only available treatment option.

Prior to 2002, Coronaviruses were not considered to be significant human pathogens. Other human Coronaviruses such as HCoV-229E and HCoV-0C43 resulted in only mild respiratory infections in healthy adults. In 2002, however, severe acute respiratory syndrome Coronavirus (SARS-CoV) emerged in Guangdong Province, China. This virus rapidly spread to 29 different countries, resulting in 8,273 confirmed cases and 775 (9%) deaths). While SARS-CoV predominantly impacted Southeast Asia, with significant outbreaks throughout China, Hong Kong, Taiwan, Singapore, and Vietnam, the virus was carried outside the region. Importation of the virus into Canada resulted in 251 confirmed cases and 44 deaths.

The emergences of SARS-CoV, MERS-CoV and recently COVID-19 have demonstrated the importance of Coronaviruses as emerging human pathogens with no effective treatment.

Viruses are dependent on their hosts for replication and dispersal in the environment; thus, the most successful viruses are those that co-evolve with their hosts. CXCR4 is a cellular chemokine receptor that plays central roles in development, hematopoiesis, and immune surveillance through signaling induced by its ligand, CXCL12. The CXCR4-CXCL12 axis has been besieged by many pathogens that employ a range of strategies to modify or exploit CXCR4 activity.

While CXCR4 was identified as a critical co-factor for entry of HIV into CD4+ T cells early on, other viruses may utilize CXCR4 to gain cell entry as well. Moreover, several viruses have been found to modulate CXCR4 expression or alter its functional activity, with direct effects on cell trafficking, immune responses, cell proliferation, and cell survival.

CXCR4 also plays a central role in modulating infiltration of neutrophils and macrophages to the site of infection, a common cause for acute respiratory distress syndrome (ARDS). Specifically, pre-clinical studies have shown that neutrophils and macrophages are one of the key cells in the pathophysiology of ARDS and acute lung injury (ALI), and are caused by various conditions. Once released, they are recruited to lung tissue where they release reactive oxygen (ROS) and nitrogen species (RNS); cationic proteins, such as myeloperoxidase (MPO); lipid mediators; inflammatory cytokines; and elastase and matrix metalloproteinases. Although these molecules are toxic to invading pathogens, they also promote epithelial and endothelial damage.¹ In addition, post-mortem examinations of the lungs of patients with malaria associated ARDS/ALI have shown the presence of pulmonary edema, inflammatory infiltrates and accumulated inflammatory cells, including neutrophils, in the interstitial and alveolar spaces.² Emerging data from autopsy samples from the lungs of COVID-19 patients observed neutrophil infiltration in pulmonary capillaries, acute capillaritis with fibrin deposition, extravasation of neutrophils into the alveolar space, and neutrophilic mucositis.³ The CXCR4/CXCL12 axis is critical in regulating the release of neutrophils from the bone marrow and their trafficking to the lungs during ARDS.⁴

Additional Related Background Art:

U.S. Pat. No. 8,663,651

U.S. Patent Application No. 20110262386.

### SUMMARY OF THE INVENTION

According to an aspect of some embodiments of the present invention there is provided a method of treating acute respiratory distress syndrome (ARDS) in a subject in need thereof, the method comprising administering to the subject a therapeutically effective amount of a CXCR4 inhibitor, thereby treating ARDS, wherein the ARDS is not associated with a bacterial or fungal infection.

According to an aspect of some embodiments of the present invention there is provided a CXCR4 inhibitor for use in treating acute respiratory distress syndrome (ARDS) in a subject in need thereof, wherein the ARDS is not associated with a bacterial or fungal infection.

According to some embodiments of the invention, the ARDS is associated with a viral infection.

According to some embodiments of the invention, the viral infection is from a virus selected from the group consisting of Influenza, Coronoviridae and Herpesviridae.

According to some embodiments of the invention, the ARDS is not associated with sepsis.

According to some embodiments of the invention, the ARDS is associated with a medical condition selected from the group consisting of barotrauma (volutrauma), pulmonary embolism (PE), ventilator-associated pneumonia (VAP), gastrointestinal: bleeding (ulcer), dysmotility, aspiration, vascular injury, pneumothorax (by placing pulmonary artery catheter), tracheal injury/stenosis (result of intubation and/or irritation by endotracheal tube), blood clots, inhalational lung injury, lung contusion, chest trauma, near-drowning, trauma (e.g. fat embolism), cardiopulmonary bypass, burns, viral infection.

According to an aspect of some embodiments of the present invention there is provided a method of treating a subject having a medical condition selected from the group consisting of barotrauma, pulmonary embolism (PE), ventilator-associated pneumonia (VAP), gastrointestinal: bleeding, dysmotility, aspiration, vascular injury, pneumothorax, tracheal injury/stenosis, blood clots, inhalational lung injury, lung contusion, chest trauma, near-drowning, trauma, cardiopulmonary bypass and burns, the method comprising administering to the subject a therapeutically effective amount of a CXCR4 inhibitor, thereby treating the medical condition in the subject.

According to an aspect of some embodiments of the present invention there is provided a method of treating Coronavirus infection, the method comprising administering to a subject in need thereof a therapeutically effective amount of a CXCR4 inhibitor to thereby treat the Coronavirus infection.

According to an aspect of some embodiments of the present invention there is provided a CXCR4 inhibitor for use in the treatment of a Coronavirus infection.

According to some embodiments of the invention, the Coronavirus is COVID-19, Middle East respiratory syndrome Coronavirus or severe acute respiratory syndrome Coronavirus.

According to some embodiments of the invention, the method further comprises administering a therapeutically effective amount of an anti-viral drug.

According to some embodiments of the invention, the use further comprises a therapeutically effective amount of an anti-viral drug.

According to some embodiments of the invention, the antiviral drug is selected from the group consisting of an interferon, chloroquine, ribavirin, adefovir, tenofovir, acyclovir, brivudin, cidofovir, fomivirsen, foscarnet, ganciclovir, penciclovir, amantadine, rimantadine and zanamivir.

According to some embodiments of the invention, the CXCR4 inhibitor is a peptide, a small molecule, an antibody, a nucleic acid or a combination of same.

According to some embodiments of the invention, the CXCR4 inhibitor is a peptide.

According to some embodiments of the invention, the peptide is as set forth in SEQ ID NO: 1 or an analog of same.

According to some embodiments of the invention, the CXCR4 inhibitor is a small molecule.

According to some embodiments of the invention, the small molecule is AMD3100.

According to some embodiments of the invention, the subject exhibits inflammation as determined by at least one marker selected from the group consisting of CRP, fibrinogen, ferritin, Di-Dimer, procalcitonin, IL6, IL-8, IL-10, IL1ra, hMPO, angiopoietin 2, RAGE, t-plasminogen and SERPIN E1.

According to some embodiments of the invention, the peptide set forth in SEQ ID NO: 1 is administered subcutaneously.

According to some embodiments of the invention, the peptide set forth in SEQ ID NO: 1 is administered at a dose of 0.5-5 mg/kg.

According to some embodiments of the invention, the peptide set forth in SEQ ID NO: 1 is administered at a dose of 0.5-2.5 mg/kg.

According to some embodiments of the invention, the peptide set forth in SEQ ID NO: 1 is administered at a dose of 0.75-1.5 mg/kg.

According to some embodiments of the invention, the peptide set forth in SEQ ID NO: 1 is administered at a dose of 1.25 mg/kg.

According to some embodiments of the invention, the peptide set forth in SEQ ID NO: 1 is administered at a daily regimen for up to 10 days.

According to some embodiments of the invention, the peptide set forth in SEQ ID NO: 1 is administered at a daily regimen for up to 7 days.

According to some embodiments of the invention, the peptide set forth in SEQ ID NO: 1 is administered at a daily regimen for 7-10 days.

According to some embodiments of the invention, the subject is infected with SARS-CoV-2, influenza, respiratory syncytial virus (RSV) or human metapneumovirus (hMP).

According to some embodiments of the invention, the effective amount causes a favorable difference in PaO₂/FIO₂ at day 10.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### DESCRIPTION OF SPECIFIC EMBODIMENTS OF THE INVENTION

The present invention, in some embodiments thereof, relates to the use of CXCR4 inhibitors for the treatment of acute respiratory distress syndrome and viral infections.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The novel Coronavirus (2019-nCov) outbreak, which initially began in China, has spread to many countries around the globe, with the number of confirmed cases increasing every day. With a death toll exceeding that of the SARS-CoV outbreak back in 2002 and 2003 in China, 2019-nCoV has led to a public health emergency of international concern, putting all health organizations on high alert.

The present inventor has now conceived the use of CXCR4 inhibitors for the treatment of Coronavirus infections.

Despite a critical role of CXCR4 in mediating neutrophil release from the bone marrow to the peripheral blood, the present inventor suggests that antagonizing the CXCR4/CXCL12 axis can be used in treating ARDS and related diseases.

In fact, the present inventor suggests a novel treatment modality of ARDS which is superficially beneficial in non-bacterial/fungal infection (where one would want to maintain the activity of macrophages and neutrophils), by inhibiting the CXCR4/CXCL12 axis. Without being bound by theory it is suggested that the net effect on inhibiting neutrophils and macrophages in situ is increased with respect to the effect on mobilization.

Thus, according to an aspect there is provided a method of treating acute respiratory distress syndrome (ARDS) in a subject in need thereof, the method comprising administering to the subject a therapeutically effective amount of a CXCR4 inhibitor, thereby treating ARDS, wherein said ARDS is not associated with a bacterial or fungal infection.

According to an aspect there is provided a CXCR4 inhibitor for use in treating acute respiratory distress syndrome (ARDS) in a subject in need thereof, wherein said ARDS is not associated with a bacterial or fungal infection.

As used herein “Acute respiratory distress syndrome (ARDS)” is a respiratory failure characterized by rapid onset (2 hours to 3 days) of widespread inflammation in the lungs. Symptoms include shortness of breath, rapid breathing, and bluish skin coloration.

Adult diagnosis is based on a PaO₂/FiO₂ ratio (ratio of partial pressure arterial oxygen and fraction of inspired oxygen) of less than 300 mm Hg despite a positive end-expiratory pressure (PEEP) of more than 5 cm H₂O. Heart-related pulmonary edema, as the cause, is to be excluded.

Adults: PaO₂/FiO₂ ratio of less than 300 mm Hg^{[1]} Children: oxygenation index>4.

According to a specific embodiment, ARDS is a result of a medical condition or trauma selected from the group consisting of barotrauma (e.g., volutrauma), pulmonary embolism (PE), ventilator-associated pneumonia (VAP), gastrointestinal: bleeding (e.g., ulcer), dysmotility, aspiration, vascular injury, pneumothorax (e.g., by placing pulmonary artery catheter), tracheal injury/stenosis (e.g., a result of intubation and/or irritation by endotracheal tube), blood clots, inhalational lung injury, lung contusion, chest trauma, near-drowning, trauma (e.g. fat embolism), cardiopulmonary bypass, burns, viral infection.

According to a specific embodiment, the ARDS is not associated with sepsis.

According to a specific embodiment, the ARDS is associated with a viral infection.

According to a specific embodiment, the viral infection is from a virus selected from the group consisting of Influenza (e.g., H1N1 and H5N1), Coronoviridae (e.g. listed hereinbelow) and Herpesviridae (e.g., herpes simplex virus (HSV) and cytomegalovirus (CMV)).

According to a specific embodiment, the virus is of a Coronaviridae.

It will be appreciated that the present teachings can be harnessed towards the treatment or prevention of a cytokine storm syndrome.

“Astorm syndrome”, also referred to as “cytokine storm”, “cytokine release syndrome” or “inflammatory cascade”, as used herein refers to the systemic inflammatory condition involving elevated levels of circulating cytokines, causing immune-cell hyperactivation, and typically leading to multisystem organ dysfunction and/or failure which can lead to death. Often, a cytokine storm is referred to as being part of a sequence or cascade because one pro-inflammatory cytokine typically leads to the production of multiple other pro-inflammatory cytokines that can reinforce and amplify the immune response.

Diagnosis of cytokine storm syndrome can be carried out using any method known in the art, such as by a subject's physical evaluation, blood tests and imaging-based evaluation. Early symptoms of cytokine storm may include, for example, high fever, fatigue, anorexia, headache, rash, diarrhea, arthralgia, myalgia, and neuropsychiatric symptoms, or any combination thereof. However, early symptoms may quickly (e.g. within hours or within days) turn into more severe and life-threating symptoms. Accordingly, subjects having cytokine storm syndrome typically have respiratory symptoms, including cough and tachypnea that can progress to acute respiratory distress syndrome (ARDS), with hypoxemia that may require mechanical ventilation. Severe symptoms of cytokine storm may include, for example, uncontrollable hemorrhaging, severe metabolism dysregulation, hypotension, cardiomyopathy, tachycardia, dyspnea, fever, ischemia or insufficient tissue perfusion, kidney failure, liver injury acute liver injury or cholestasis, multisystem organ failure, or any combination thereof. Blood tests typically illustrate hyperinflammation as measured, for example, by C-reactive protein (CRP) levels, and blood-count abnormalities, such as leukocytosis, leukopenia, anemia, thrombocytopenia, and elevated ferritin and d-dimer levels.

According to one embodiment, cytokine storm syndrome is typically associated with elevated serum levels of at least 40%, at least 50%, at least 60%, at least 70%, e.g. at least 50% (compared to basal state) of one or more cytokine, such as but not limited to, IFN-α, IFN-γ, TNF-α, IL-1 (e.g. IL-1α, IL-1β), IL-2, IL-5, IL-6, IL-7, IL-12, IL-178, IL-18, IL-21, IL-17, IL-33 and HMGB-1, or chemokine, such as but not limited to, IL-8, MIG, IP-10, MCP-1 (e.g., MIP-1α, MIP-1β), and BLC. Assessment of cytokine levels can be carried out using any method known in the art, such as but not limited to, by ELISA or immunoassay.

According to one embodiment, the subject may be a subject at any stage of the cytokine storm, e.g. a subject showing preliminary signs of a cytokine storm (e.g. elevated CRP levels, elevated cytokine levels, having early symptoms of cytokine storm as discussed above), a subject showing mild signs of cytokine storm (e.g. showing signs of organ dysfunction, requiring oxygen, blood tests showing hyperinflammation), a subject having severe signs of cytokine storm (e.g. requiring mechanical ventilation, hemorrhaging, having multisystem organ dysfunction and/or failure) or a subject after the severe stage of a cytokine storm.

Cytokine storms can be triggered by various pathogens, therapies, cancers, autoimmune and autoinflammatory conditions, and monogenic disorders, as further discussed below.

According to one embodiment, the cytokine storm syndrome is associated with an infectious disease.

According to a specific embodiment, the cytokine storm is viral-induced.

Viral infectious diseases commonly associated with a cytokine storm include, but at not limited to, malaria, avian influenza, smallpox, pandemic influenza, adult respiratory distress syndrome (ARDS), severe acute respiratory syndrome (SARS). According to one embodiment, the infectious agents include, but are not limited to, Ebola, Marburg, Crimean-Congo hemorrhagic fever (CCHF), South American hemorrhagic fever, dengue, yellow fever, Rift Valley fever, Omsk hemorrhagic fever virus, Kyasanur Forest, Junin, Machupo, Sabia, Guanarito, Garissa, Ilesha, or Lassa fever viruses. According to one embodiment, the viral infectious agents include, but are not limited to, coronavirus, rhinovirus, paramyxoviridae, Orthomyxoviridae, adenovirus, parainfluenza virus, metapneumovirus, respiratory syncytial virus, influenza virus, Epstein-Barr virus, cytomegalovirus, flavivirus, variola and hantavirus.

According to one embodiment, the cytokine storm is induced by a virus causing a respiratory infection, such as but not limited to, influenza virus or coronavirus.

According to one embodiment, the cytokine storm is induced by a coronavirus. Exemplary coronaviruses include, but are not limited to, severe acute respiratory syndrome coronavirus 2 (SARS-CoV-2), a Middle East respiratory syndrome coronavirus (MERS-CoV) and a severe acute respiratory syndrome coronavirus (SARS-CoV). Additional examples are provided herein below.

According to one embodiment, the cytokine storm is induced by an influenza virus. Exemplary influenza viruses include, but are not limited to, H1N1 (Spanish influenza) and H5N1 (Avian flu).

According to one embodiment, the cytokine storm is bacterial-induced. Exemplary bacterial pathogens which can induce a cytokine storm include, but are not limited to, streptococcus species (e.g. streptococcus group A) and Staphylococcus aureus.

According to one embodiment, the cytokine storm syndrome is associated with a medical condition, such as acute respiratory distress syndrome.

According to one embodiment, the cytokine storm syndrome is lung-associated.

According to one embodiment, the cytokine storm syndrome is airway-associated.

According to an aspect of the invention, there is provided a method of treating Coronavirus infection, the method comprising administering to a subject in need thereof a therapeutically effective amount of a CXCR4 inhibitor to thereby treat the Coronavirus infection.

According to another aspect there is provided a CXCR4 inhibitor for use in the treatment of a Coronavirus infection.

As used herein “Coronavirus” refers to enveloped positive-stranded RNA viruses that belong to the family Coronaviridae and the order Nidovirales.

Examples of Corona viruses which are contemplated herein include, but are not limited to, 229E, NL63, OC43, and HKU1 with the first two classified as antigenic group 1 and the latter two belonging to group 2, typically leading to an upper respiratory tract infection manifested by common cold symptoms.

Commercial multiplex PCR assays for respiratory pathogens may detect these viruses. It is important that positive results for these viruses should not be confused with MERS-CoV.

However, Coronaviruses, which are zoonotic in origin, can evolve into a strain that can infect human beings leading to fatal illness. Thus particular examples of Coronaviruses contemplated herein are SARS-CoV, MERS-CoV, and SARS-CoV-2 causing the recently identified 2019-nCoV (also referred to as “COVID-19”).

The expansion of genetic diversity among Coronaviruses and their consequent ability to cause disease in human beings is mainly achieved through infecting peridomestic animals, which serve as intermediate hosts, nurturing recombination and mutation events.

Accordingly embodiments of the invention refer to a non-human subject.

According to another embodiment, the subject is a human subject.

According to a specific embodiment, the subject does not exhibit clinical symptoms of infection.

It would be appreciated that any Coronavirus strain is contemplated herein even though some are emphasized in a detailed manner.

Thus, a clinical manifestation of Coronavirus infection includes symptoms selected from the group consisting of inflammation in the lung, alveolar damage, fever, cough, shortness of breath, diarrhea, organ failure, pneumonia and/or septic shock.

According to a specific embodiment, the subject may not exhibit symptoms, i.e., asymptomatic carrier.

Methods of analyzing infection are well known in the art and are either based on serology, protein markers, or nucleic acid assays.

According to some embodiments, infection is based on detection of unique sequences of virus RNA by NAAT such as real-time reverse-transcription polymerase chain reaction (c-PCR) with confirmation by nucleic acid sequencing when necessary.

The term “treating” refers to inhibiting, preventing or arresting the development of a medical condition (disease, disorder or condition) and/or causing the reduction, remission, or regression of a medical condition. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

As used herein, the term “subject” includes mammals, preferably human beings at any age which suffer from the pathology. According to some embodiments, this term encompasses individuals who are at risk to develop the pathology (e.g., has been exposed or is at risk of being exposed to a respiratory viral infection.

According to a specific embodiment, the subject exhibits inflammation as determined by at least one marker selected from the group consisting of CRP, fibrinogen, ferritin, Di-Dimer, procalcitonin, IL6, IL-8, IL-10, IL1ra, hMPO, angiopoietin 2, RAGE, t-plasminogen and SERPIN E1.

According to a specific embodiment, the subject is infected with Coronavirus (e.g., SARS-CoV-2), influenza, respiratory syncytial virus (RSV) or human metapneumovirus (hMP), such as determined by RT-PCR.

As used herein, the term “CXCR4 inhibitor” refers to molecules and compositions that interfere with or inhibit the biological activity of the CXCR4 receptor. Biological activity of the CXCR4 receptor can include, entry of the virus to the cell or replication of the virus in the cell (without being bound by theory).

The CXCR4 inhibitors can encompass numerous classes of chemical molecules, e.g., small organic or inorganic molecules, polysaccharides, biological macromolecules, e.g., peptides, proteins, peptide analogs and derivatives, peptidomimetics, antibodies, antibody fragments, nucleic acids, nucleic acid analogs and derivatives such as aptamers, an extract made from biological materials such as bacteria, plants, fungi, or animal cells or tissues, naturally occurring or synthetic compositions.

Without wishing to be bound by a theory, a CXCR4 inhibitor can act by a number of different pathways. For example, a CXCR4 inhibitor can bind to a ligand bind site on the CXCR4 receptor and interfere with binding of the ligand to the CXCR4 receptor, bind to a nonligand binding site on the CXCR4 receptor and interfere with binding of the ligand to the CXCR4 receptor, bind with a CXCR4 receptor ligand and interfere with binding of the ligand to the CXCR4 receptor, or inhibit the expression of a polynucleotide (e.g., mRNA) expressing CXCR4

In some embodiments, a CXCR4 inhibitor inhibits the biological activity of the CXCR4 receptor by at least 5%, at least 10%, at least 15%, at least 20%, at least 30%, at least 40%, at least 50%, at least 60%, at least 70%, at least 75%, at least 80%, at least 85%, at least 90%, or at least 95% relative to a control. In some embodiments, a CXCR4 inhibitor completely abrogates the biological activity of the CXCR4 receptor relative to a control. A control can comprise a sample that is not treated an inhibitor.

In some embodiments, a CXCR4 inhibitor is a nucleic acid. Exemplary CXCR4 nucleic acid inhibitors include, but are not limited to, antisense oligonucleotides, siRNAs, shRNAs, microRNAs, aptamers, ribozymes and decoy oligonucleotides. A CXCR4 nucleic acid inhibitor can inhibit the expression of a CXCR4 gene.

Exemplary anti CXCR4 siRNAs are described, for example, in U.S. Pat. App. Pub. No. 2007/0238868, No. 2009/0253772, content of both of which is incorporated herein by reference. Some exemplary CXCR4 antisense oligonucleotides are described, for example, in U.S. Pat. App. Pub. No. 2004/0209837, content of which is incorporated herein by reference.

In some embodiments, the CXCR4 inhibitor binds to CXCR4 or to CXCL12 (SDF-1 alpha). In another embodiment, the CXCR4 inhibitor is an antibody or antibody fragment. In some embodiments, the CXCR4 inhibitor is a small molecule, for example, AMD-3100, ALX40-4C, T22, T140, Met-SDF1beta, T134, or AMD-3465.

Exemplary CXCR4 inhibitors include, but are not limited to, 2,T-bicyclam; 6,6′-bicyclam; the embodiments set forth in U.S. Pat. Nos. 5,021,409, and 6,001,826, and in particular 1,1′-[1,4-phenylene-bis(methylene)]-bis-1,4,8,11tetraazacyclotetradecane, set forth in U.S. Pat. No. 5,583,131, and designated herein AMD3100. In some embodiments, a CXCR4 inhibitor can be N′-(1Hbenzimidazol-2-yl methyl)-N′-(5,6,7,8-tetrahydroquinoline8-yl)-butane-1,4-diamine as described in U.S. Patent Publication No. 2003/0220341, CTCF-0214; CTCF-9908; CP-1221 (linear peptides, cyclic peptides, natural amino-acids, unnatural amino acids, and peptidomimetic compounds); 4F-benzoylTN24003; KRH-1120; KRH-1636; KRH-2731; polyphemusin analogue; ALX40-4C; or those described in WO 01/85196; WO 99/50461; WO 01/94420; WO 03/090512, each of which is incorporated by reference herein in its entirety.

In some embodiments, CXCR4 inhibitors include the T-140 analogs and antibodies described in US Patent Publication 2010/0055088, the cycle polyamines described in US Patent Publication 2009/0221683, and the compounds disclosed in US Patent Publication Nos. 2004/0209921, 2005/0059702, 2005/0043367, 2005/0277670, 2010/0178271, and 2003/0220341; U.S. Pat. Nos. 5,021,409, 6,001,826, 5,583,131, and Patent Publication WO 03/011277, each of which are incorporated herein by reference in their entirety.

CXCR4 inhibitors can also include, but are not limited to, polypeptides that specifically bind to CXCR4. Such inhibitors include T140 and derivatives of T140. Exemplary derivatives of T140 include, but are not limited to, TN14003, TC14012, and TE14011 as well as those found in Tamamura, H. et al. Org. Biomol. Chem. 1:3656-3662, 2003, which is incorporated by reference herein in its entirety.

According to specific embodiments, the CXCR4-antagonistic peptides of the present invention are for example, 4F-benzoyl-TN14003 (SEQ ID NO: 1) analogs and derivatives and are structurally and functionally related to the peptides disclosed in patent applications WO 2002/020561 and WO 2004/020462, also known as “T-140 analogs”, as detailed hereinbelow.

In various particular embodiments, the T-140 analog or derivative has an amino acid sequence as set forth in the following formula (I) or a salt thereof:

1 2 3 4 5 6 7 8 9 10 11 12 13 14

A₁-A₂-A₃-Cys-Tyr-A₄-A₅-A₆-A₇-A₈-A₉-A₁₀-Cys-A₁₁ (I)

wherein:

A₁ is an arginine, lysine, ornithine, citrulline, alanine or glutamic acid residue or a N-α-substituted derivative of these amino acids, or A₁ is absent;

A₂ represents an arginine or glutamic acid residue if A₁ is present, or A₂ represents an arginine or glutamic acid residue or a N-α-substituted derivative of these amino acids if A₁ is absent;

A₃ represents an aromatic amino acid residue;

A₄, As and A₉ each independently represents an arginine, lysine, ornithine, citrulline, alanine or glutamic acid residue;

A₆ represents a proline, glycine, ornithine, lysine, alanine, citrulline, arginine or glutamic acid residue;

A₇ represents a proline, glycine, ornithine, lysine, alanine, citrulline or arginine residue;

A₈ represents a tyrosine, phenylalanine, alanine, naphthylalanine, citrulline or glutamic acid residue;

A₁₀ represents a citrulline, glutamic acid, arginine or lysine residue;

A₁₁ represents an arginine, glutamic acid, lysine or citrulline residue wherein the C-terminal carboxyl may be derivatized;

and the cysteine residue of the 4-position or the 13-position can form a disulfide bond, and the amino acids can be of either L or D form.

Exemplary peptides according to formula (I) are peptides having an amino acid sequence as set forth in any one of SEO ID NOS:1-72, as presented in Table 1 hereinbelow.

TABLE 1
T-140 and currently preferred T-140 analoks

| | SEQ | |
|---|---|---|
| Analog | ID NO: | Amino acid sequence |
| 4F-benzoyl- | 1 | 4F-benzoyl-Arg-Arg-Nal-Cys-Tyr-Cit-Lys-DLys-Pro-Tyr-Arg-Cit-Cys-Arg-NH₂ |
| TN14003 | | |
| AcTC14003 | 2 | Ac-Arg-Arg-Nal-Cys-Tyr-Cit-Lys-DLys-Pro-Tyr-Arg-Cit-Cys-Arg-OH |
| AcTC14005 | 3 | Ac-Arg-Arg-Nal-Cys-Tyr-Arg-Lys-DCit-Pro-Tyr-Arg-Cit-Cys-Arg-OH |
| AcTC14011 | 4 | Ac-Arg-Arg-Nal-Cys-Tyr-Cit-Lys-DCit-Pro-Tyr-Arg-Cit-Cys-Arg-OH |
| AcTC14013 | 5 | Ac-Arg-Arg-Nal-Cys-Tyr-Cit-Lys-DLys-Pro-Tyr-Cit-Cit-Cys-Arg-OH |
| AcTC14015 | 6 | Ac-Cit-Arg-Nal-Cys-Tyr-Cit-Lys-DLys-Pro-Tyr-Arg-Cit-Cys-Arg-OH |
| AcTC14017 | 7 | Ac-Cit-Arg-Nal-Cys-Tyr-Arg-Lys-DCit-Pro-Tyr-Arg-Cit-Cys-Arg-OH |
| AcTC14019 | 8 | Ac-Arg-Arg-Nal-Cys-Tyr-Arg-Lys-DCit-Pro-Tyr-Cit-Cit-Cys-Arg-OH |
| AcTC14021 | 9 | Ac-Cit-Arg-Nal-Cys-Tyr-Arg-Lys-DLys-Pro-Tyr-Cit-Cit-Cys-Arg-OH |
| AcTC14012 | 10 | Ac-Arg-Arg-Nal-Cys-Tyr-Cit-Lys-DCit-Pro-Tyr-Arg-Cit-Cys-Arg-NH₂ |
| AcTC14014 | 11 | Ac-Arg-Arg-Nal-Cys-Tyr-Cit-Lys-DLys-Pro-Tyr-Cit-Cit-Cys-Arg-NH₂ |
| AcTC14016 | 12 | Ac-Cit-Arg-Nal-Cys-Tyr-Cit-Lys-DLys-Pro-Tyr-Arg-Cit-Cys-Arg-NH₂ |
| AcTC14018 | 13 | Ac-Cit-Arg-Nal-Cys-Tyr-Arg-Lys-DCit-Pro-Tyr-Arg-Cit-Cys-Arg-NH₂ |
| AcTC14020 | 14 | Ac-Arg-Arg-Nal-Cys-Tyr-Arg-Lys-DCit-Pro-Tyr-Cit-Cit-Cys-Arg-NH₂ |
| AcTC14022 | 15 | Ac-Cit-Arg-Nal-Cys-Tyr-Arg-Lys-DLys-Pro-Tyr-Cit-Cit-Cys-Arg-NH₂ |
| TE14001 | 16 | H-DG1u-Arg-Nal-Cys-Tyr-Arg-Lys-DLys-Pro-Tyr-Arg-Cit-Cys-Arg-OH |
| TE14002 | 17 | H-Arg-Glu-Nal-Cys-Tyr-Arg-Lys-DLys-Pro-Tyr-Arg-Cit-Cys-Arg-OH |
| TE14003 | 18 | H-Arg-Arg-Nal-Cys-Tyr-Glu-Lys-DLys-Pro-Tyr-Arg-Cit-Cys-Arg-OH |
| TE14004 | 19 | H-Arg-Arg-Nal-Cys-Tyr-Arg-Glu-DLys-Pro-Tyr-Arg-Cit-Cys-Arg-OH |
| TE14005 | 20 | H-Arg-Arg-Na1-Cys-Tyr-Arg-Lys-DG1u-Pro-Tyr-Arg-Cit-Cys-Arg-OH |
| TE14006 | 21 | H-Arg-Arg-Nal-Cys-Tyr-Arg-Lys-DLys-Pro-Tyr-Glu-Cit-Cys-Arg-OH |
| TE14007 | 22 | H-Arg-Arg-Nal-Cys-Tyr-Arg-Lys-DLys-Pro-Tyr-Arg-Cit-Cys-Glu-OH |
| TE14011 | 23 | H-Arg-Arg-Na1-Cys-Tyr-Cit-Lys-DG1u-Pro-Tyr-Arg-Cit-Cys-Arg-NH₂ |
| TE14012 | 24 | H-Arg-Arg-Na1-Cys-Tyr-DG1u-Lys-DCit-Pro-Tyr-Arg-Cit-Cys-Arg-NH₂ |
| TE14013 | 25 | H-Arg-Arg-Na1-Cys-Tyr-DG1u-Lys-DG1u-Pro-Tyr-Arg-Cit-Cys-Arg-NH₂ |
| TE14014 | 26 | H-DG1u-Arg-Na1-Cys-Tyr-Cit-Lys-DG1u-Pro-Tyr-Arg-Cit-Cys-Arg-NH₂ |
| TE14015 | 27 | H-Arg-Arg-Na1-Cys-Tyr-Cit-Lys-DG1u-Pro-DG1u-Arg-Cit-Cys-Arg-NH₂ |
| TE14016 | 28 | H-Arg-Arg-Na1-Cys-Tyr-Cit-Lys-DG1u-Pro-Tyr-Arg-DG1u-Cys-Arg-NH₂ |
| AcTE14014 | 29 | Ac-DG1u-Arg-Nal-Cys-Tyr-Cit-Lys-DG1u-Pro-Tyr-Arg-Cit-Cys-Arg-NH₂ |
| AcTE14015 | 30 | Ac-Arg-Arg-Nal-Cys-Tyr-Cit-Lys-DG1u-Pro-DG1u-Arg-Cit-Cys-Arg-NH₂ |
| AcTE14016 | 31 | Ac-Arg-Arg-Nal-Cys-Tyr-Cit-Lys-DG1u-Pro-Tyr-Arg-DG1u-Cys-Arg-NH₂ |
| TF1: | 32 | Ac-Arg-Arg-Na1-Cys-Tyr-Cit-Lys-DG1u-Pro-Tyr-Arg-Cit-Cys-Arg-NH₂ |
| AcTE14011 | | |
| TF2: guanyl- | 33 | guanyl-Arg-Arg-Nal-Cys-Tyr-Cit-Lys-DG1u-Pro-Tyr-Arg-Cit-Cys-Arg-NH₂ |
| TE14011 | | |
| TF3: | 34 | TMguanyl-Arg-Arg-Nal-Cys-Tyr-Cit-Lys-DG1u-Pro-Tyr-Arg-Cit-Cys-Arg-NH₂ |
| TMguanyl- | | |
| TE14011 | | |
| TF4: | 35 | TMguanyl-Arg-Nal-Cys-Tyr-Cit-Lys-DG1u-Pro-Tyr-Arg-Cit-Cys-Arg-NH₂ |
| TMguanyl- | | |
| TE14011 (2-14) | | |
| TF5: 4F- | 36 | 4F-benzoyl-Arg-Arg-Nal-Cys-Tyr-Cit-Lys-DG1u-Pro-Tyr-Arg-Cit-Cys-Arg-NH₂ |
| benzoyl- | | |
| TE14011 | | |
| TF6: 2F- | 37 | 2F-benzoyl-Arg-Arg-Nal-Cys-Tyr-Cit-Lys-DG1u-Pro-Tyr-Arg-Cit-Cys-Arg-NH₂ |
| benzoyl- | | |
| TE14011 | | |
| TF7: APA- | 38 | APA-Arg-Na1-Cys-Tyr-Cit-Lys-DG1u-Pro-Tyr-Arg-Cit-Cys-Arg-NH₂ |
| TE14011 (2-14) | | |
| TF8: desamino- | 39 | desamino-R-Arg-Nal-Cys-Tyr-Cit-Lys-DG1u-Pro-Tyr-Arg-Cit-Cys-Arg-NH₂ |
| R-TE14011 (2-14) | | |
| TF9: guanyl- | 40 | Guanyl-Arg-Na1-Cys-Tyr-Cit-Lys-DG1u-Pro-Tyr-Arg-Cit-Cys-Arg-NH₂ |
| TE14011 (2-14) | | |
| TF10: succinyl- | 41 | succinyl-Arg-Nal-Cys-Tyr-Cit-Lys-DG1u-Pro-Tyr-Arg-Cit-Cys-Arg-NH₂ |
| TE14011 (2-14) | | |
| TF11: glutaryl- | 42 | glutaryl-Arg-Nal-Cys-Tyr-Cit-Lys-DG1u-Pro-Tyr-Arg-Cit-Cys-Arg-NH₂ |
| TE14011 (2-14) | | |
| TF12: | 43 | deaminoTMG-APA-Arg-Nal-Cys-Tyr-Cit-Lys-DG1u-Pro-Tyr-Arg-Cit-Cys-Arg-NH₂ |
| deaminoTMG- | | |
| APA-TE14011 | | |
| (2-14) | | |
| TF15: H-Arg- | 44 | R-CH2-Arg-Na1-Cys-Tyr-Cit-Lys-DG1u-Pro-Tyr-Arg-Cit-Cys-Arg-NH₂ |
| CH2NH- | | |
| RTE14011 (2-14) | | |
| TF17: TE14011 | 45 | H-Arg-Na1-Cys-Tyr-Cit-Lys-DG1u-Pro-Tyr-Arg-Cit-Cys-Arg-NH₂ |
| (2-14) | | |
| TF18: | 46 | TMguanyl-Arg-Arg-Nal-Cys-Tyr-Cit-Lys-DCit-Pro-Tyr-Arg-Cit-Cys-Arg-NH₂ |
| TMguanyl- | | |
| TC14012 | | |
| TF19: ACA- | 47 | ACA-Arg-Arg-Nal-Cys-Tyr-Cit-Lys-DCit-Pro-Tyr-Arg-Cit-Cys-Arg-NH₂ |
| TC14012 | | |
| TF20: ACA- | 48 | ACA-Arg-Arg-Nal-Cys-Tyr-Arg-Lys-DLys-Pro-Tyr-Arg-Cit-Cys-Arg-OH |
| T140 | | |
| TZ14011 | 49 | H-Arg-Arg-Na1-Cys-Tyr-Cit-Arg-DLys-Pro-Tyr-Arg-Cit-Cys-Arg-NH₂ |
| AcTZ14011 | 50 | Ac-Arg-Arg-Na1-Cys-Tyr-Cit-Arg-DLys-Pro-Tyr-Arg-Cit-Cys-Arg-NH₂ |
| AcTN14003 | 51 | Ac-Arg-Arg-Na1-Cys-Tyr-Cit-Lys-DLys-Pro-Tyr-Arg-Cit-Cys-Arg-NH₂ |
| AcTN14005 | 52 | Ac-Arg-Arg-Na1-Cys-Tyr-Arg-Lys-DCit-Pro-Tyr-Arg-Cit-Cys-Arg-NH₂ |
| 4F-benzoyl- | 53 | 4F-benzoyl-Arg-Arg-Nal-Cys-Tyr-Cit-Lys-DG1u-Pro-Tyr-Arg-Cit-Cys-Arg- |
| TN14011-Me | | NHMe |
| 4F-benzoyl- | 54 | 4F-benzoyl-Arg-Arg-Nal-Cys-Tyr-Cit-Lys-DG1u-Pro-Tyr-Arg-Cit-Cys-Arg- |
| TN14011-Et | | NHEt |
| 4F-benzoyl- | 55 | 4F-benzoyl-Arg-Arg-Nal-Cys-Tyr-Cit-Lys-DG1u-Pro-Tyr-Arg-Cit-Cys-Arg- |
| TN14011-iPr | | NHiPr |
| 4F-benzoyl- | 56 | 4F-benzoyl-Arg-Arg-Nal-Cys-Tyr-Cit-Lys-DG1u-Pro-Tyr-Arg-Cit-Cys-Arg- |
| TN14011- | | tyramine |
| tyramine | | |
| TA14001 | 57 | H-Ala-Arg-Nal-Cys-Tyr-Arg-Lys-DLys-Pro-Tyr-Arg-Cit-Cys-Arg-OH |
| TA14005 | 58 | H-Arg-Arg-Nal-Cys-Tyr-Ala-Lys-DLys-Pro-Tyr-Arg-Cit-Cys-Arg-OH |
| TA14006 | 59 | H-Arg-Arg-Nal-Cys-Tyr-Arg-Ala-DLys-Pro-Tyr-Arg-Cit-Cys-Arg-OH |
| TA14007 | 60 | H-Arg-Arg-Nal-Cys-Tyr-Arg-Lys-DAla-Pro-Tyr-Arg-Cit-Cys-Arg-OH |
| TA14008 | 61 | H-Arg-Arg-Nal-Cys-Tyr-Arg-Lys-DLys-Ala-Tyr-Arg-Cit-Cys-Arg-OH |
| TA14009 | 62 | H-Arg-Arg-Nal-Cys-Tyr-Arg-Lys-DLys-Pro-Ala-Arg-Cit-Cys-Arg-OH |
| TA14010 | 63 | H-Arg-Arg-Nal-Cys-Tyr-Arg-Lys-DLys-Pro-Tyr-Ala-Cit-Cys-Arg-OH |
| TC14001 | 64 | H-Cit-Arg-Nal-Cys-Tyr-Arg-Lys-DLys-Pro-Tyr-Arg-Cit-Cys-Arg-OH |
| TC14003 | 65 | H-Arg-Arg-Nal-Cys-Tyr-Cit-Lys-DLys-Pro-Tyr-Arg-Cit-Cys-Arg-OH |
| TN14003 | 66 | H-Arg-Arg-Na1-Cys-Tyr-Cit-Lys-DLys-Pro-Tyr-Arg-Cit-Cys-Arg-NH₂ |
| TC14004 | 67 | H-Arg-Arg-Nal-Cys-Tyr-Arg-Cit-DLys-Pro-Tyr-Arg-Cit-Cys-Arg-OH |
| TC14012 | 68 | H-Arg-Arg-Na1-Cys-Tyr-Cit-Lys-DCit-Pro-Tyr-Arg-Cit-Cys-Arg-NH₂ |
| T-140 | 69 | H-Arg-Arg-Nal-Cys-Tyr-Arg-Lys-DLys-Pro-Tyr-Arg-Cit-Cys-Arg-OH |
| TC14011 | 70 | H-Arg-Arg-Nal-Cys-Tyr-Cit-Lys-DCit-Pro-Tyr-Arg-Cit-Cys-Arg-OH |
| TC14005 | 71 | H-Arg-Arg-Nal-Cys-Tyr-Arg-Lys-DCit-Pro-Tyr-Arg-Cit-Cys-Arg-OH |
| TC14018 | 72 | H-Cit-Arg-Na1-Cys-Tyr-Arg-Lys-DCit-Pro-Tyr-Arg-Cit-Cys-Arg-NH₂ |

According to a specific embodiment, in each one of SEQ ID NOS:1-72, two cysteine residues are coupled in a disulfide bond.

In another embodiment, the analog or derivative has an amino acid sequence as set forth in SEQ ID NO:65 (H-Arg-Arg-Nal-Cys-Tyr-Cit-Lys-DLys-Pro-Tyr-Arg-Cit-Cys-Arg-OH; TC14003).

In another embodiment, the peptide used in the compositions and methods of the invention consists essentially of an amino acid sequence as set forth in SEQ ID NO:1. In another embodiment, the peptide used in the compositions and methods of the invention comprises an amino acid sequence as set forth in SEQ ID NO:1. In another embodiment, the peptide is at least 60%, at least 70% or at least 80% homologous to SEQ ID NO:1. In another embodiment, the peptide is at least 90% homologous to SEQ ID NO:1. In another embodiment, the peptide is at least about 95% homologous to SEQ ID NO: 1. Each possibility represents a separate embodiment of the present invention.

In various other embodiments, the peptide is selected from SEQ ID NOS:1-72, wherein each possibility represents a separate embodiment of the present invention.

In another embodiment, the peptide has an amino acid sequence as set forth in any one of SEQ ID NOS: 1-4, 10, 46, 47, 51-56, 65, 66, 68, 70 and 71. In another embodiment, the peptide has an amino acid sequence as set forth in any one of SEQ ID NOS: 4, 10, 46, 47, 68 and 70. In another embodiment, the peptide has an amino acid sequence as set forth in any one of SEQ ID NOS:1, 2, 51, 65 and 66. In another embodiment, the peptide has an amino acid sequence as set forth in any one of SEQ ID NOS:53-56.

In an embodiment, the peptide has an amino acid sequence as set forth in SEQ ID NO:1. In another embodiment, the peptide has an amino acid sequence as set forth in SEQ ID NO:2. In another embodiment, the peptide has an amino acid sequence as set forth in SEQ ID NO:51. In another embodiment, the peptide has an amino acid sequence as set forth in SEQ ID NO:66.

Other CXCR4 peptide inhibitors (antagonists) include but are not limited to LY2510924 (by Lilly Oncology), CTCE-9908 (Huang et al. 2009 Journal of Surgical Research 155:231-236), Fc131 analogs and nanobodies as specified in the citations below (each of which is incorporated herein by reference in its entirety):
Tan N C, Yu P, Kwon Y-U, Kodadek T. High-throughput evaluation of relative cell permeability between peptoids and peptides. Bioorg Med Chem. 2008; 16:5853-61.
Kwon Y-U, Kodadek T. Quantitative evaluation of the relative cell permeability of peptoids and peptides. J Am Chem Soc. 2007; 129:1508.
Miller S, Simon R, Ng S, Zuckermann R, Kerr J, Moos W. Comparison of the proteolytic susceptibilities of homologous L-amino acid, D-amino acid, and N-substituted glycine peptide and peptoid oligomers. Drug Dev Res. 1995; 35:20-32.
Yoshikawa Y, Kobayashi K, Oishi S, Fujii N, Furuya T. Molecular modeling study of cyclic pentapeptide CXCR4 antagonists: new insight into CXCR4-FC131 interactions. Bioorg Med Chem Lett. 2012; 22:2146-50.
Jaähnichen S, Blanchetot C, Maussang D, Gonzalez-Pajuelo M, Chow K Y, Bosch L, De Vrieze S, Serruys B, Ulrichts H, Vandevelde W. CXCR4 nanobodies (VHH-based single variable domains) potently inhibit chemotaxis and HIV-1 replication and mobilize stem cells. Proc Natl Acad Sci USA. 2010; 107:20565-70.

The CXCR4 antagonist (e.g., SEQ ID NO: 1) of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the CXCR4 antagonist (e.g., SEQ ID NO: 1) accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common Coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (CXCR4 antagonist (e.g., SEQ ID NO: 1)) effective to prevent, alleviate or ameliorate symptoms of a disorder (e.g., Coronavirus infection) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

With respect to COVID-19, early reports suggest that the virus can utilize human, bat, swine, and civet ACE2.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide therapeutic levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

According to a specific embodiment, the peptide set forth in SEQ ID NO: 1 is administered subcutaneously.

According to a specific embodiment, the peptide set forth in SEQ ID NO: 1 is administered at a dose of 0.5-5 mg/kg.

According to a specific embodiment, the peptide set forth in SEQ ID NO: 1 is administered at a dose of 0.5-2.5 mg/kg.

According to a specific embodiment, the peptide set forth in SEQ ID NO: 1 is administered at a dose of 0.75-1.5 mg/kg.

According to a specific embodiment, the peptide set forth in SEQ ID NO: 1 is administered at a dose of 1.25 mg/kg.

According to a specific embodiment, the peptide set forth in SEQ ID NO: 1 is administered at a daily regimen for up to 21 days.

According to a specific embodiment, the peptide set forth in SEQ ID NO: 1 is administered at a daily regimen for up to 14 days.

According to a specific embodiment, the peptide set forth in SEQ ID NO: 1 is administered at a daily regimen for up to 10 days.

According to a specific embodiment, the peptide set forth in SEQ ID NO: 1 is administered at a daily regimen for up to 7 days.

According to a specific embodiment, the peptide set forth in SEQ ID NO: 1 is administered at a daily regimen for 7-10 days.

According to a specific embodiment, the peptide set forth in SEQ ID NO: 1 is administered at a daily regimen for 5-20 days.

According to a specific embodiment, the peptide set forth in SEQ ID NO: 1 is administered at a daily regimen for 5-14 days.

According to a specific embodiment, the peptide set forth in SEQ ID NO: 1 is administered at a daily regimen for 5-10 days.

According to a specific embodiment, the peptide set forth in SEQ ID NO: 1 is administered at a daily regimen for 5-8 days.

According to a specific embodiment, the peptide set forth in SEQ ID NO: 1 is administered at a daily regimen for 7 days.

According to a specific embodiment, the effective amount causes a favorable difference in PaO₂/FIO₂ such as at day 10 following initiation of treatment.

According to a specific embodiment, treatment is combined with Gold standard therapies including, but not limited to mechanical ventilation together with treatments directed at the underlying cause. Ventilation strategies include using low volumes and low pressures. If oxygenation remains insufficient, lung recruitment maneuvers and neuromuscular blockers may be used. If these are insufficient, extracorporeal membrane oxygenation (ECMO).

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The present teachings further envisage treating with other anti-viral drugs or anti-inflammatory drugs or anti-coagulants as separate treatments or in a co-formulation.

According to a specific embodiment, the antiviral drug is selected from the group consisting of remdesivir, an interferon, ribavirin, adefovir, tenofovir, acyclovir, brivudin, cidofovir, fomivirsen, foscarnet, ganciclovir, penciclovir, amantadine, rimantadine and zanamivir.

Also contemplated are plasma treatments from infected persons who survived and/or anti-HIV drugs such as lopinavir and ritonavir, as well as chloroquine.

Specific examples for drugs that are routinely used for the treatment of COVID-19 include, but are not limited to, Lopinavir/Ritonavir, Nucleoside analogues, Neuraminidase inhibitors, Remdesivir, peptide (EK1), abidol, RNA synthesis inhibitors (such as TDF, 3TC), anti-inflammatory drugs (such as hormones and other molecules), Chinese traditional medicine, such ShuFengJieDu Capsules and Lianhuaqingwen Capsule, could be the drug treatment options for 2019-nCoV.

According to other embodiments, the CXCR4 inhibitors is combined with another medication selected from the group consisting of Actmera (Tocilizumab), Remdesivir, Baricitinib (e.g. such as in combination with Remdesivir), Dexamethasone, Anticoagulation drugs (e.g., Clexane, Eliquis (apixaban)), Nexium (esomeprazole), Proton-pump inhibitors (PPIs), Tavanic (Levofloxacin), Acetylcysteine, Inhaled Corticosteroid (ICS), Aerovent, Solvex (Bromhexine Hydrochloride), Sopa K (Potassium gluconate), Chloroquine (e.g. Hydroxychloroquine), Antibiotic (e.g. Azenil/Azithromycin/Zitromax, Amoxicillin/Moxypen Forte, Ceftriaxone/Rocephin).

It is expected that during the life of a patent maturing from this application many relevant CXCR4 inhibitors will be developed and the scope of the term CXCR4 inhibitor is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

### Cytopathic Endpoint Assay

The protocol used is adapted from Al-Jabri et al. (Virology methods manual. London: Academic Press Ltd; 1996. p. 293-356), and the peptide set forth SEQ ID NO: 1 is tested in quadruplicate. Briefly, 100 μL of serial 10-fold dilutions of the peptide is incubated with 100 μL of Vero E6 cells, giving a final cell count of 20,000 cells per well in a 96-well plate. The incubation period is 1 h at 37° C. in 5% CO₂, except for the interferons, which are incubated overnight with the cells. Ten μL of virus (e.g., SARS or COVID-19) at a concentration of 10,000 PFU/well is then added to each of the test wells. The plates are incubated at 37° C. in 5% CO₂ for 3 days and observed daily for CPE. The end point is the peptide dilution that inhibited 100% of the CPE (CIA₁₀₀) in quadruplicate wells. To determine cytotoxicity, 100 μL of serial 10-fold dilutions of the peptide is incubated with 100 μL of Vero E6 cells, giving a final cell count of 20,000 cells per well in a 96-well plate, without viral challenge. The plates are then incubated at 37° C. in 5% CO₂ for 3 days and examined for toxicity effects by using an inverted microscope.

### Example 2

### Clinical Protocol

Aim: To study the safety and efficacy of BL-8040 (A CXCR4 antagonist, set forth in SEQ ID NO: 1) on top of standard of treatment for patients with acute respiratory distress syndrome (ARDS) due to respiratory viral infections.

Protocol Synopsis:

| | |
|---|---|
| Study title | A Phase Ib, open-label pilot study designed to |
| | evaluate the safety and efficacy of BL-8040 (a CXCR4 antagonist) |
| | on top of standard of treatment for patients with acute respiratory |
| | distress syndrome (ARDS) due to respiratory viral infections |
| Protocol no. | |
| Clinical sites | Wolfson Medical Center, Holon, Israel |
| Study phase | Ib |
| Therapeutic | ARDS due to respiratory viral infections |
| indication | |
| Study Objectives | Primary |
| | To assess the safety of BL-8040 in patients with ARDS secondary to |
| | viral infections. |
| | Secondary |
| | To assess the change in PaO₂/FIO₂ in response to treatment with BL- |
| | 8040 in patients with ARDS due to respiratory viral infections at day |
| | 10. |
| | To assess the clinical efficacy of the treatment with BL-8040 in |
| | patients with ARDS due to respiratory viral infections. |
| | To assess the time to response following treatment with BL-8040 in |
| | patients with ARDS due to respiratory viral infections. |
| | To assess the change in blood inflammatory markers in response to |
| | treatment with BL-8040 in patients with ARDS due to respiratory |
| | viral infections. |
| | Exploratory |
| | To assess ventilation days in response to treatment with BL-8040 in |
| | patients with ARDS due to respiratory viral infections. |
| Study Design | This will be an open-label, single arm, phase Ib study in subjects with |
| | ARDS due to COVID-19 or other viral respiratory agents. |
| | Eligible subjects will receive subcutaneous (SC) injections of BL-8040, |
| | an anti CXCR4 antagonist over 7 days. |
| | The primary objective of the study is to determine the safety and |
| | tolerability of the treatment with BL-8040 on top of the standard |
| | treatment (antiviral and other supportive care). |
| | Safety and efficacy will be assessed at defined time-points throughout |
| | the study. |
| | A maximum of 25 patients will be enrolled in the study. |
| Study | Screening Period (Visit 1, Day −1 to Day 0) |
| Procedures | Adult male and female subjects aged 18-85 will be screened for study |
| | eligibility by assessment of inclusion and exclusion criteria. |
| | The following assessments are to be performed at Screening visit 1: |
| | Complete demographics including disease related medical history |
| | and general medical history. |
| | Concomitant medications. |
| | Complete physical examination. |
| | Weight for BL-8040 dosing. |
| | Viral etiology of ARDS as confirmed by PCR |
| | Vital signs (blood pressure, pulse rate, temperature, ventilation |
| | parameters, PaO₂/FIO₂). |
| | SOFA score and APACHE score. |
| | Inflammatory parameters (CRP, fibrinogen, ferritin, Di-Dimer, |
| | procalcitonin, IL6, IL-8, IL-10, IL1ra, hMPO, angiopoietin 2, |
| | RAGE, t-plasminogen, SERPIN E1). |
| | Blood count, biochemistry, coagulation (Pro-thrombin time [PT] and |
| | aPTT) and serum pregnancy test (for women of child-bearing |
| | potential. |
| | Chest X-ray. |
| | Patients will receive the first dose of BL-8040 within 48 hours of |
| | screening. |
| | Treatment period |
| | Treatment period is 7 days. |
| | BL-8040 will be administered at a dose of 1.25 mg/kg SC on Days 1 to 7. |
| | Assessments during study period (up to 30 days after the first dose) |
| | Vital signs (blood pressure, pulse rate, temperature, ventilation |
| | parameters, PaO₂/FIO₂) - daily up to day 10 and on day 15, 20, 25, |
| | 30, or until weaning from ventilation (first of the two). |
| | SOFA score and APACHE score on day 1, 5, 7, 10, 20, 30 or until |
| | weaning from ventilation (first of the two). |
| | Laboratory safety evaluations (minimal requirements) on days 1, 3, |
| | 5, 7 - blood count, biochemistry, coagulation (Pro-thrombin time |
| | [PT] and aPTT). |
| | Blood count - daily before BL-8040 administration. |
| | On days 5, 7, 10 - Inflammatory parameters (CRP, fibrinogen, |
| | ferritin, Di-Dimer, procalcitonin, IL6, IL-8, IL-10, IL1ra, hMPO, |
| | angiopoietin 2, RAGE, t-plasminogen, SERPIN E1). |
| | Adverse events will be assessed and recorded throughout the study |
| | up to and including 30 days from the last dose of BL-8040. |
| | Concomitant Medications will be recorded throughout the study. |
| | 12-lead ECG 1-24 hours before administrating the first dose; 1 hour |
| | after the administration on Day 1. |
| | Efficacy assessment |
| | Change in PaO₂/FIO₂ at day 10 from baseline. |
| | Secondary |
| | Change from baseline in blood inflammatory markers in response to |
| | treatment with BL-8040 on day 5, 7, 10. |
| | Exploratory |
| | Ventilation days and time to weaning from ventilation. |
| Study Duration | 30 days |
| | Screening - 1 day |
| | Treatment - 7 days |
| | Test of cure - on day 10 |
| | Follow up - 30 days |
| Planned sample | 25 patients will be enrolled in the study. An interim analysis will be |
| size | conducted after a maximum of 10 patients have completed treatment and |
| | a joint decision will be taken on the continuation of enrollment. |
| Inclusion | All of the following: |

| | | |
|---|---|---|
| Criteria | 1. | Adult men and women aged 18-85. |
| | 2. | Informed consent by a guardian or an independent physician |
| | | (according to IRB approval). |
| | 3. | Respiratory viral infection diagnosed by a positive PCR from a |
| | | respiratory sample (nasal, or deep suction aspirate) to one of the |
| | | following pathogens: SARS-CoV-2, influenza, respiratory |
| | | syncytial virus (RSV), human metapneumovirus (hMP). |
| | 4. | A chest X-ray or CT-scan compatible with the diagnosis of |
| | | ARDS. |
| | 5. | Requiring mechanical ventilation. |
| | 6. | PaO₂/FIO₂ below 350 mmHg. |
| | 7. | Adequate organ function at Baseline as defined below: |

| | |
|---|---|
| a. | Hematological: |

| | |
|---|---|
| i. | White blood cell (WBC) ≥2,500/mm\hat{ }3 |
| ii. | Absolute neutrophil count: ≥1000 /mm\hat{ }3 |
| iii. | Platelet count ≥100,000/mm\hat{ }3 |
| iv. | Hemoglobin ≥9 g/dL or ≥5.6 mmol/L |
| v. | Hematocrit ≥30% |

| | |
|---|---|
| b. | Renal function: |

| | |
|---|---|
| i. | Creatinine ≤1.5x Upper limit of normal (ULN) |
| | OR measured or calculated creatinine clearance |
| | (glomerular filtration rate [GFR]) can also be |
| | used in place of creatinine or (CrCl) >60 |
| | mL/min for subject with creatinine levels >1.5x |
| | institutional ULN |

| | |
|---|---|
| c. | Hepatic function: |

| | |
|---|---|
| i. | Total Bilirubin: within institutional normal |
| | ranges |
| ii. | Aspartate aminotransferase/serum glutamic |
| | oxaloacetic transaminase (AST/SGOT) and |
| | Alanine amino transferase/serum glutamate- |
| | pyruvate transaminase (ALT/SGPT): ≤2.5xULN |

| | |
|---|---|
| d. | Coagulation: |

| | |
|---|---|
| i. | INR or PT: ≤1.5xULN unless subject is |
| | receiving anticoagulant therapy as long as PT or |
| | PTT is within therapeutic range of intended use |
| | of anticoagulants |
| ii. | aPTT: ≤1.5xULN unless subject is receiving |
| | anticoagulant therapy as long as PT or PTT is |
| | within therapeutic range of intended use of |
| | anticoagulants |

| Exclusion | Any of the following: | |
|---|---|---|
| Criteria | 1. | Known allergy or hypersensitivity to any of the test compounds, |
| | | materials or contraindication to BL-8040. |
| | 2. | More than 48 hours from the beginning of mechanical ventilations. |
| | 3. | Has a disease that is suitable for therapy administered with curative |
| | | intent. |
| | 4. | Intracranial hypertension. |
| | 5. | Undrained pneumothorax or subcutaneous emphysema. |
| | 6. | Has a positive HIV test at Screening or at any time prior to |
| | | Screening. Patients without a prior positive HIV test result will |
| | | undergo an HIV test at Screening, unless not permitted per local |
| | | regulations. |
| | 7. | Has known active Hepatitis B (defined as having a positive Hepatitis |
| | | B surface antigen (HBsAg) test at Screening) or Hepatitis C (defined |
| | | as having a positive HCV antibody test or a positive HCV RNA test |
| | | at Screening). |
| | 8. | Has known history of Chronic Hepatitis B or C. |
| | 9. | Patients who have had any major surgical procedure within 30 days |
| | | of Day 1. |
| | 10. | Patient with any concomitant malignant disease. |
| | 11. | Is pregnant or breastfeeding or expecting to conceive or father |
| | | children within the projected duration of the trial, starting with the |
| | | Screening visit through 120 days after the last dose of trial |
| | | treatment. Women with a positive pregnancy test within 72 hours |
| | | from Baseline. |
| | 12. | Life expectancy of ≤2 months |

| | |
|---|---|
| Investigational | BL-8040, a synthetic polypeptide highly selective antagonist of CXCR4. |
| Product Route | BL-8040, a white to off-white powder, is freely soluble in water. It is |
| and Dosage | manufactured in accordance with Good Manufacturing Practice (cGMP) |
| | by BioConnection B.V. (previously MSD), Kloosterstraat 9, 5349 AB |
| | Oss, Netherlands. BL-8040 (1.25 mg/kg) will be administrated by |
| | subcutaneous (SC) injections daily on Days 1-7. |
| Safety and | Safety will be evaluated based on the following parameters: |
| Tolerability | General safety: vital signs (oral temperature, blood pressure, pulse rate, |
| | respiratory rate and O2 saturation), 12-lead ECG and physical |
| | examination. |
| Statistical | The primary objective of the study is to determine the safety and |
| Analysis | tolerability in patients with ARDS due to respiratory viruses. |
| | A maximum of 25 patients will be enrolled in the study. |
| | Safety monitoring |
| | The method of Thall, Simon and Estey will be used for toxicity |
| | monitoring for this study. Denote the probability of toxicity by p(T), |
| | where toxicity is defined as BL-8040 treatment-related, clinically |
| | significant Grade 3 or higher nonhematologic clinically significant |
| | toxicity that occurs anytime during the treatment. Exceptions: ≥Grade 3 |
| | nausea, vomiting, hair loss, constipation, rigors, chills, hemorrhage, or |
| | any other condition as judged by the investigator as clinically |
| | insignificant. |
| | We will stop the trial for new patient enrollment if at any time during the |
| | study there is more than 80% chance that the toxicity rate is more than |
| | 30%. This toxicity-stopping rule will be applied by cohort size of 5, |
| | starting from the 5th patient. |
| | Statistical analysis |
| | All patients who received at least one dose of the study drag will be |
| | included in the intent-to-treat analysis (ITT) for efficacy and safety. |
| | Demographic/clinical characteristics and safety data of the patients will |
| | be summarized using descriptive statistics such as mean, standard |
| | deviation, median and range. Details about the data analysis will be |
| | provided within the Statistical Analysis Plan. |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited Throughout the Specification

1. Jochen Grommes Contribution of Neutrophils to Acute Lung Injury Mol Med. 2011 March-April; 17(3-4): 293-307. Published online 2010 Oct. 18. doi: 10.2119/molmed.2010.00138
2. Sercundes M K, Ortolan L S, Debone D, Soeiro-Pereira P V, Gomes E, et al. (2017) Correction: Targeting Neutrophils to Prevent Malaria-Associated Acute Lung Injury/Acute Respiratory Distress Syndrome in Mice. PLOS Pathogens 13(11): e1006730. www(dot)doi(dot)org/10(dot)1371/journal(dot)ppat(dot)1006730
3. Betsy J. Barnes; Targeting potential drivers of COVID-19: Neutrophil extracellular traps J Exp Med (2020) 217 (6): e20200652. www(dot)doi(dot)org/10(dot)1084/jem(dot)20200652
4. Katia De Filippo Sara M. Rankin; CXCR4, the master regulator of neutrophil trafficking in homeostasis and disease Eur J Clin Invest. 2018; 48 (Suppl. 2):
5. Shaabani, N., Zak, J., Johnson, ISG15 drives immune pathology and respiratory failure during viral infection doi: www(dot)doi(dot)org/10(dot)1101/2020(dot)04(dot)13(dot)039321
6. Miriam Merad and Jerome C. Martin; Pathological inflammation in patients with COVID-19: a key role for monocytes and macrophages Nature Reviews Immunology (2020)
7. Pieter Ruytinx, Paul Proost, Jo Van Damme and Sofie Struyf; Chemokine-Induced Macrophage Polarization in Inflammatory conditions Frontiers in Immunology 2 Sep. 2018|Volume 9|Article 1930
8. Tian, X., Xie, G., Xiao, H. et al. CXCR4 knockdown prevents inflammatory cytokine expression in macrophages by suppressing activation of MAPK and NF-κB signaling pathways. Cell Biosci 9, 55 (2019). www(dot)doi(dot)org/10(dot)1186/s13578-019-0315-x Lukacs N W, Berlin A, Schols D, Skerlj R T, Bridger G J.
9. Lukacs N W, Berlin A, Schols D, Skerlj R T, Bridger G J. AMD3100, a CxCR4 antagonist, attenuates allergic lung inflammation and airway hyperreactivity. Am J Pathol. American Society for Investigative Pathology; 2002; 160: 1353±60. doi: 10.1016/S0002-9440(10)62562-X PMID: 11943720
10. Drummond S, Ramachandran S, Torres E, Huang J, Hehre D, Suguihara C, et al. CXCR4 blockade attenuates hyperoxia-induced lung injury in neonatal rats. Neonatology. 2015; 107: 304±311. doi: 10.1159/000371835 PMID: 25825119
11. Devi S, Wang Y, Chew W K, Lima R, N A G, Mattar C N, et al. Neutrophil mobilization via plerixafor-mediated CXCR4 inhibition arises from lung margination and blockade of neutrophil homing to the bone marrow. J Exp Med. 2013; 210: 2321±2336. doi: 10.1084/jem.20130056 PMID: 24081949
12. Sercundes M K, Ortolan L S, Debone D, Soeiro-Pereira P V, Gomes E, et al. (2017) Correction: Targeting Neutrophils to Prevent Malaria-Associated Acute Lung Injury/Acute Respiratory Distress Syndrome in Mice. PLOS Pathogens 13(11): e1006730. www(dot)doi(dot)org/10(dot)1371/journal(dot)ppat(dot)1006730
13. Yamada M, Kubo H, Kobayashi S, Ishizawa K, He M, Suzuki T, et al. The increase in surface CXCR4 expression on lung extravascular neutrophils and its effects on neutrophils during endotoxin-induced lung injury. Cell Mol Immunol. Nature Publishing Group; 2011; 8: 305±314. doi: 10.1038/cmi.2011.8 PMID: 21460863.

What is claimed is:

1. A method of treating acute respiratory distress syndrome (ARDS) in a subject in need thereof, the method comprising administering to the subject a therapeutically effective amount of a CXCR4 inhibitor, thereby treating ARDS, and wherein said ARDS is associated with a viral infection selected from the group consisting of Influenza, Coronoviridae and Herpesviridae.

2. A method of treating Coronavirus infection, the method comprising administering to a subject in need thereof a therapeutically effective amount of a CXCR4 inhibitor to thereby treat the Coronavirus infection, wherein said treating is not vaccination.

3. The method of claim 2, wherein said Coronavirus is SARS-Cov-2, Middle East respiratory syndrome Coronavirus or severe acute respiratory syndrome Coronavirus.

4. The method of claim 1, wherein said CXCR4 inhibitor is a peptide, a small molecule, an antibody, a nucleic acid or a combination of same.

5. The method of claim 2, wherein said CXCR4 inhibitor is a peptide, a small molecule, an antibody, a nucleic acid or a combination of same.

6. The method of claim 4, wherein said CXCR4 inhibitor is a peptide.

7. The method of claim 5, wherein said CXCR4 inhibitor is a peptide.

8. The method of claim 6, wherein said peptide is as set forth in SEQ ID NO: 1.

9. The method of claim 7, wherein said peptide is as set forth in SEQ ID NO: 1.

10. The method of claim 4, wherein said CXCR4 inhibitor is a small molecule.

11. The method of claim 10, wherein said small molecule is AMD3100.

12. The method of claim 5, wherein said CXCR4 inhibitor is a small molecule.

13. The method of claim 12, wherein said small molecule is AMD3100.

14. The method of claim 1, wherein the subject exhibits inflammation as determined by at least one marker selected from the group consisting of CRP, fibrinogen, ferritin, Di-Dimer, procalcitonin, IL6, IL-8, IL-10, IL1ra, hMPO, angiopoietin 2, RAGE, t-plasminogen and SERPIN E1.

15. The method of claim 8, wherein said peptide set forth in SEQ ID NO: 1 is administered at a dose of 0.75-1.5 mg/kg.

16. The method of claim 9, wherein said peptide set forth in SEQ ID NO: 1 is administered at a dose of 0.75-1.5 mg/kg.


SEQUENCE LISTING (72 sequences)

SEQ ID NO: 1 - protein, 14 aa, Artificial
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): C′ AMIDATED
XRXCYXKXPYRXCR

SEQ ID NO: 2 - protein, 14 aa, Artificial
  SYNTHETIC PEPTIDE
  MOD_RES (1)..(1): N′ ACETYLATION
  MOD_RES (3)..(3): 3-((2-naphthyl) alanine
  MOD_RES (6)..(6): citrulline
  MOD_RES (8)..(8): D-lysine
  MOD_RES (12)..(12): citrulline
RRXCYXKXPYRXCR

SEQ ID NO: 3 - protein, 14 aa, Artificial
  SYNTHETIC PEPTIDE
  MOD_RES (1)..(1): N′ ACETYLATION
  MOD_RES (3)..(3): 3-((2-naphthyl) alanine
  MOD_RES (8)..(8): D-citrulline
  MOD_RES (12)..(12): citrulline
RRXCYRKXPYRXCR

SEQ ID NO: 4 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-citrulline
  MOD_RES (12)..(12): citrulline
RRXCYXKXPYRXCR

SEQ ID NO: 5 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-lysine
  MOD_RES (11)..(12): citrulline
RRXCYXKXPYXXCR

SEQ ID NO: 6 - protein, 14 aa, Artificial
  synthetic peptide
  MOD_RES (1)..(1): N′ acetylated citrulline
  MOD_RES (3)..(3): 3-((2-naphthyl) alanine
  MOD_RES (6)..(6): citrulline
  MOD_RES (8)..(8): D-lysine
  MOD_RES (12)..(12): citrulline
XRXCYXKXPYRXCR

SEQ ID NO: 7 - protein, 14 aa, Artificial
  synthetic peptide
  MOD_RES (1)..(1): N′ acetylated citrulline
  MOD_RES (3)..(3): 3-((2-naphthyl) alanine
  MOD_RES (8)..(8): D-citrulline
  MOD_RES (12)..(12): citrulline
XRXCYRKXPYRXCR

SEQ ID NO: 8 - protein, 14 aa, Artificial
  synthetic peptide
  MOD_RES (1)..(1): N′ ACETYLATED
  MOD_RES (3)..(3): 3-((2-naphthyl) alanine
  MOD_RES (8)..(8): D-citrulline
  MOD_RES (11)..(12): citrulline
RRXCYRKXPYXXCR

SEQ ID NO: 9 - protein, 14 aa, Artificial
  synthetic peptide
  MOD_RES (1)..(1): N′ ACETYLATED citrulline
  MOD_RES (3)..(3): 3-((2-naphthyl) alanine
  MOD_RES (8)..(8): D-lysine
  MOD_RES (11)..(12): citrulline
XRXCYRKXPYXXCR

SEQ ID NO: 10 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-citrulline
  MOD_RES (12)..(12): citrulline
  MOD_RES (14)..(14): C′ AMIDATED
RRXCYXKXPYRXCR

SEQ ID NO: 11 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-lysine
  MOD_RES (11)..(12): citrulline
  MOD_RES (14)..(14): C′ AMIDATED
RRXCYXKXPYXXCR

SEQ ID NO: 12 - protein, 14 aa, Artificial
  synthetic peptide
  MOD_RES (1)..(1): N′ ACETYLATED citrulline
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): AMIDATED
XRXCYXKXPYRXCR

SEQ ID NO: 13 - protein, 14 aa, Artificial
  synthetic peptide
  MOD_RES (1)..(1): N′ ACETYLATED citrulline
  MOD_RES (3)..(3): 3-((2-naphthyl) alanine
  MOD_RES (8)..(8): D-citrulline
  MOD_RES (12)..(12): citrulline
  MOD_RES (14)..(14): C′ AMIDATED
XRXCYRKXPYRXCR

SEQ ID NO: 14 - protein, 14 aa, Artificial
  synthetic peptide
  MOD_RES (1)..(1): N′ ACETYLATED
  MOD_RES (3)..(3): 3-((2-naphthyl) alanine
  MOD_RES (8)..(8): D-citrulline
  MOD_RES (11)..(12): citrulline
  MOD_RES (14)..(14): C′ AMIDATED
RRXCYRKXPYXXCR

SEQ ID NO: 15 - protein, 14 aa, Artificial
  synthetic peptide
  MOD_RES (1)..(1): N′ ACETYLATED citrulline
  MOD_RES (3)..(3): 3-((2-naphthyl) alanine
  MOD_RES (8)..(8): D-lysine
  MOD_RES (11)..(12): citrulline
  MOD_RES (14)..(14): C′ AMIDATED
XRXCYRKXPYXXCR

SEQ ID NO: 16 - protein, 14 aa, Artificial
  synthetic peptide
  MOD_RES (1)..(1): D-glutamic acid
  MOD_RES (3)..(3): 3-((2-naphthyl) alanine
  MOD_RES (8)..(8): D-lysine
  MOD_RES (12)..(12): citrulline
XRXCYRKXPYRXCR

SEQ ID NO: 17 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
REXCYRKXPYRXCR

SEQ ID NO: 18 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYEKXPYRXCR

SEQ ID NO: 19 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYREXPYRXCR

SEQ ID NO: 20 - protein, 14 aa, Artificial
  synthetic peptide
  MOD_RES (3)..(3): 3-((2-naphthyl) alanine
  MOD_RES (8)..(8): D-glutamic acid
  MOD_RES (12)..(12): citrulline
RRXCYRKXPYRXCR

SEQ ID NO: 21 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRKXPYEXCR

SEQ ID NO: 22 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRKXPYRXCE

SEQ ID NO: 23 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): C′ amidated
RRXCYXKXPYRXCR

SEQ ID NO: 24 - protein, 14 aa, Artificial
  synthetic peptide
  MOD_RES (3)..(3): 3-((2-naphthyl) alanine
  MOD_RES (6)..(6): D-glutamic acid
  MOD_RES (8)..(8): D-citrulline
  MOD_RES (12)..(12): citrulline
  MOD_RES (14)..(14): c′ amidated
RRXCYXKXPYRXCR

SEQ ID NO: 25 - protein, 14 aa, Artificial
  synthetic peptide
  MOD_RES (3)..(3): 3-((2-naphthyl) alanine
  MOD_RES (6)..(6): D-glutamic acid
  MOD_RES (8)..(8): D-glutamic acid
  MOD_RES (12)..(12): citrulline
  MOD_RES (14)..(14): c′ amidated
RRXCYXKXPYRXCR

SEQ ID NO: 26 - protein, 14 aa, Artificial
  synthetic peptide
  MOD_RES (1)..(1): D-glutamic acid
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 27 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-glutamic acid
  MOD_RES (10)..(10): D-glutamic acid
  MOD_RES (12)..(12): citrulline
  MOD_RES (14)..(14): c′ amidated
RRXCYXKXPXRXCR

SEQ ID NO: 28 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-glutamic acid
  MOD_RES (12)..(12): D-glutamic acid
  MOD_RES (14)..(14): c′ amidated
RRXCYXKXPYRXCR

SEQ ID NO: 29 - protein, 14 aa, Artificial
  synthetic peptide
  MOD_RES (1)..(1): N′ ACETYLATED D-glutamic acid
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 30 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-glutamic acid
  MOD_RES (10)..(10): D-glutamic acid
  MOD_RES (12)..(12): citrulline
  MOD_RES (14)..(14): c′ amidated
RRXCYXKXPXRXCR

SEQ ID NO: 31 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-glutamic acid
  MOD_RES (12)..(12): D-glutamic acid
  MOD_RES (14)..(14): c′ amidated
RRXCYXKXPYRXCR

SEQ ID NO: 32 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
RRXCYXKXPYRXCR

SEQ ID NO: 33 - protein, 14 aa, Artificial
  synthetic peptide
  MOD_RES (1)..(1): Guanyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 34 - protein, 14 aa, Artificial
  synthetic peptide
  MOD_RES (1)..(1): Tetramethylguanyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 35 - protein, 13 aa, Artificial
  synthetic peptide
  MOD_RES (1)..(1): Tetramethylguanyl-arginine
  MOD_RES (2)..(2): 3-((2-naphthyl) alanine
  MOD_RES (5)..(5): citrulline
  MOD_RES (7)..(7): D-glutamic acid
  MOD_RES (11)..(11): citrulline
  MOD_RES (13)..(13): c′ amidated
XXCYXKXPYRXCR

SEQ ID NO: 36 - protein, 14 aa, Artificial
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): amidated
XRXCYXKXPYRXCR

SEQ ID NO: 37 - protein, 14 aa, Artificial
  synthetic peptide
  MOD_RES (1)..(1): 2-fluorobenzoyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 38 - protein, 13 aa, Artificial
  synthetic peptide
  MOD_RES (1)..(1): 5-aminopentanoyl-arginine
  MOD_RES (2)..(2): 3-((2-naphthyl) alanine
  MOD_RES (5)..(5): citrulline
  MOD_RES (7)..(7): D-glutamic acid
  MOD_RES (11)..(11): citrulline
  MOD_RES (13)..(13): c′ amidated
XXCYXKXPYRXCR

SEQ ID NO: 39 - protein, 14 aa, Artificial
  synthetic peptide
  MOD_RES (1)..(1): 2-desamino-arginyl
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 40 - protein, 13 aa, Artificial
  synthetic peptide
  MOD_RES (1)..(1): Guanyl-arginine
  MOD_RES (2)..(2): 3-((2-naphthyl) alanine
  MOD_RES (5)..(5): citrulline
  MOD_RES (7)..(7): D-glutamic acid
  MOD_RES (11)..(11): citrulline
  MOD_RES (13)..(13): c′ amidated
XXCYXKXPYRXCR

SEQ ID NO: 41 - protein, 13 aa, Artificial
  synthetic peptide
  MOD_RES (1)..(1): Succinyl-arginine
  MOD_RES (2)..(2): 3-((2-naphthyl) alanine
  MOD_RES (5)..(5): citrulline
  MOD_RES (7)..(7): D-glutamic acid
  MOD_RES (11)..(11): citrulline
  MOD_RES (13)..(13): c′ amidated
XXCYXKXPYRXCR

SEQ ID NO: 42 - protein, 13 aa, Artificial
  synthetic peptide
  MOD_RES (1)..(1): Glutaryl-arginine
  MOD_RES (2)..(2): 3-((2-naphthyl) alanine
  MOD_RES (5)..(5): citrulline
  MOD_RES (7)..(7): D-glutamic acid
  MOD_RES (11)..(11): citrulline
  MOD_RES (13)..(13): c′ amidated
XXCYXKXPYRXCR

SEQ ID NO: 43 - protein, 14 aa, Artificial
  synthetic peptide
  MISC_FEATURE (1)..(1): desaminoTMG-APA (formula IV in the specification)
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 44 - protein, 14 aa, Artificial
  synthetic peptide
  MISC_FEATURE (1)..(1): R-CH2 - formula (V) in the specification
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 45 - protein, 13 aa, Artificial
  synthetic peptide
  MOD_RES (2)..(2): 3-((2-naphthyl) alanine
  MOD_RES (5)..(5): citrulline
  MOD_RES (7)..(7): D-glutamic acid
  MOD_RES (11)..(11): citrulline
  MOD_RES (13)..(13): c′ amidated
RXCYXKXPYRXCR

SEQ ID NO: 46 - protein, 14 aa, Artificial
  synthetic peptide
  MOD_RES (1)..(1): tetramethylguanyl-arginine
  MOD_RES (3)..(3): 3-((2-naphthyl) alanine
  MOD_RES (6)..(6): citrulline
  MOD_RES (8)..(8): D-citrulline
  MOD_RES (12)..(12): citrulline
  MOD_RES (14)..(14): C′ AMIDATED
XRXCYXKXPYRXCR

SEQ ID NO: 47 - protein, 14 aa, Artificial
  synthetic peptide
  MOD_RES (1)..(1): 6-aminohexanoyl-arginine
  MOD_RES (3)..(3): 3-((2-naphthyl) alanine
  MOD_RES (6)..(6): citrulline
  MOD_RES (8)..(8): D-citrulline
  MOD_RES (12)..(12): citrulline
  MOD_RES (14)..(14): C′ AMIDATED
XRXCYXKXPYRXCR

SEQ ID NO: 48 - protein, 14 aa, Artificial
  synthetic peptide
  MOD_RES (1)..(1): 6-aminohexanoyl-arginine
  MOD_RES (3)..(3): 3-((2-naphthyl) alanine
  MOD_RES (8)..(8): D-lysine
  MOD_RES (12)..(12): citrulline
XRXCYRKXPYRXCR

SEQ ID NO: 49 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): C′ amidated
RRXCYXRXPYRXCR

SEQ ID NO: 50 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): C′ amidated
RRXCYXRXPYRXCR

SEQ ID NO: 51 - protein, 14 aa, Artificial
  SYNTHETIC PEPTIDE
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): C′ AMIDATED
RRXCYXKXPYRXCR

SEQ ID NO: 52 - protein, 14 aa, Artificial
  synthetic peptide
  MOD_RES (1)..(1): N′ ACETYLATED
  MOD_RES (3)..(3): 3-((2-naphthyl) alanine
  MOD_RES (8)..(8): D-citrulline
  MOD_RES (12)..(12): citrulline
  MOD_RES (14)..(14): C′ AMIDATED
RRXCYRKXPYRXCR

SEQ ID NO: 53 - protein, 14 aa, Artificial
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MISC_FEATURE (14)..(14): derivatization by a NH-methyl group
XRXCYXKXPYRXCR

SEQ ID NO: 54 - protein, 14 aa, Artificial
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MISC_FEATURE (14)..(14): derivatization by a NH-ethyl group
XRXCYXKXPYRXCR

SEQ ID NO: 55 - protein, 14 aa, Artificial
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MISC_FEATURE (14)..(14): derivatization by NH-isopropyl
XRXCYXKXPYRXCR

SEQ ID NO: 56 - protein, 14 aa, Artificial
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MISC_FEATURE (14)..(14): derivatization with a tyramine residue
XRXCYXKXPYRXCR

SEQ ID NO: 57 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
ARXCYRKXPYRXCR

SEQ ID NO: 58 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYAKXPYRXCR

SEQ ID NO: 59 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRAXPYRXCR

SEQ ID NO: 60 - protein, 14 aa, Artificial
  synthetic peptide
  MOD_RES (3)..(3): 3-((2-naphthyl) alanine
  MOD_RES (8)..(8): D-alanine
  MOD_RES (12)..(12): citrulline
RRXCYRKXPYRXCR

SEQ ID NO: 61 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRKXAYRXCR

SEQ ID NO: 62 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRKXPARXCR

SEQ ID NO: 63 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRKXPYAXCR

SEQ ID NO: 64 - protein, 14 aa, Artificial
  synthetic peptide
  MOD_RES (1)..(1): citrulline
  MOD_RES (3)..(3): 3-((2-naphthyl) alanine
  MOD_RES (8)..(8): D-lysine
  MOD_RES (12)..(12): citrulline
XRXCYRKXPYRXCR

SEQ ID NO: 65 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-lysine
  MOD_RES (12)..(12): citrulline
RRXCYXKXPYRXCR

SEQ ID NO: 66 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): C′ AMIDATED
RRXCYXKXPYRXCR

SEQ ID NO: 67 - protein, 14 aa, Artificial
  synthetic peptide
  MOD_RES (3)..(3): 3-((2-naphthyl) alanine
  MOD_RES (7)..(7): citrulline
  MOD_RES (8)..(8): D-lysine
  MOD_RES (12)..(12): citrulline
RRXCYRXXPYRXCR

SEQ ID NO: 68 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-citrulline
  MOD_RES (12)..(12): citrulline
  MOD_RES (14)..(14): C′ AMIDATED
RRXCYXKXPYRXCR

SEQ ID NO: 69 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRKXPYRXCR

SEQ ID NO: 70 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-citrulline
  MOD_RES (12)..(12): citrulline
RRXCYXKXPYRXCR

SEQ ID NO: 71 - protein, 14 aa, Artificial
  synthetic peptide
  MOD_RES (3)..(3): 3-((2-naphthyl) alanine
  MOD_RES (8)..(8): D-citrulline
  MOD_RES (12)..(12): citrulline
RRXCYRKXPYRXCR

SEQ ID NO: 72 - protein, 14 aa, Artificial
  synthetic peptide
  MOD_RES (1)..(1): citrulline
  MOD_RES (3)..(3): 3-((2-naphthyl) alanine
  MOD_RES (8)..(8): D-citrulline
  MOD_RES (12)..(12): citrulline
  MOD_RES (14)..(14): C′ Amidated
XRXCYRKXPYRXCR